(19) United States
(12) Patent Application Publication
(10) Pub. No.: US 2024/0027452 A1
(43) Pub. Date: Jan. 25, 2024

(54) METHODS, DEVICES AND SYSTEMS FOR DETECTION OF BIOMARKERS

(71) Applicant: MIG USA, LLC, High Point, NC (US)

(72) Inventor: Adnan Mjalli, High Point, NC (US)

(21) Appl. No.: 18/268,478

(22) PCT Filed: Dec. 21, 2021
(86) PCT No.: PCT/US21/64602

§ 371 Date: Jun. 20, 2023

Related U.S. Application Data
(60) Provisional application No. 63/128,578, filed on Dec. 21, 2020.
(60) Provisional application No. 63/129,409, filed on Dec. 22, 2020.

(51) Int. Cl. G01N 33/569; G01N 33/543

(52) U.S. Cl. CPC G01N 33/56983; G01N 33/5438; G01N 2333/165; G01N 2469/10

(57) ABSTRACT

Disclosed are methods, systems and devices for detection of biomarkers. In certain embodiments, the methods and/or devices and/or systems may be used for the detection of biomarkers characteristic of disease. For example, disclosed are methods, systems and devices that may be used to detect and distinguish a biomarker profile indicative of the presence of COVID-19 as either an active infection, or a subject in remission, or a subject who has not been exposed to the virus.

### RELATED APPLICATIONS

The present application claims priority to U.S. Provisional Application Nos. 63/128,578 filed Dec. 21, 2020 and 63/048,449 filed Dec. 22, 2021. U.S. Provisional Application Nos. 63/128,578 and 63/048,449 are incorporated by reference in their entireties herein.

### FIELD OF THE INVENTION

The present disclosure relates to methods, devices, and systems for detection of biomarkers.

### BACKGROUND

In recent years there has been an increasing understanding that the human organism responds in a very similar way to disorders of different natures, trauma, exposure to infectious or non-infectious pathogens, and the risks of disease through the release of a cascade inflammatory mediators, which produce a response proportional to the magnitude of the disorder or disease. Thus, through detection of these mediators it is possible to determine early diagnosis of disorders such as lupus, rheumatoid arthritis, endometriosis and Alzheimer's disease, among others. Additionally, inflammatory mediators and viral antigens can be used to detect disease resulting from viral infections, including Coronavirus Disease 2019 (COVID-19). COVID-19 is a respiratory disease caused by severe acute respiratory syndrome coronavirus 2 (SARS-CoV-2). Coronaviruses (CoV) are a large family of viruses that cause human illness ranging from the common cold to more severe diseases, such as Middle East Respiratory Syndrome (MERS) and Severe Acute Respiratory Syndrome (SARS).

As a precedent, the conventional ELISA (Enzyme linked Immunosorbent Assay) test method is an enzyme immunoassay capable of detecting and quantifying cytokine and chemokine proteins that can be classified into four types: direct, indirect, sandwich, and competition. In summary, ELISA utilizes immobilized antibodies or antigens on a substrate to detect a marker of interest (e.g. antibodies, antigens, proteins and glycoproteins). Soluble markers present in a sample or standard can specifically bind to the immobilized antibodies or antigens, which allows for detection and quantitation.

The ELISA method is commonly use in the art for the detection of biomarkers due to its high specificity and sensitivity, high efficiency, safety, and ecological soundness due to its use of organic solvents and lack of radioactive reagents. However, this method has several disadvantages, for example, it is laborious, there is often a high possibility of false negatives and false positives, and the assay is often high in cost due to the use of antibodies. Generation of antibodies can require sophisticated techniques and transportation and storage can be expensive due to antibody instability. For example, antibodies must be refrigerated at the correct temperature, so that protein denaturation does not occur.

Another technique used for detecting biomarkers of interest is flow cytometry. Flow cytometry is a multiparameter analysis technique that allows for the measurement of multiple physical and chemical characteristics in single samples with applicability in several areas such as immunology, virology, molecular biology and monitoring of infectious diseases. Flow cytometry works by means of one or more lasers that pass through a sample of cells suspended in solution where the particles are analyzed according to the detection of light scattering or fluorescence. Despite the wide applicability, flow cytometry has some disadvantages, including the use of a cell suspension, which may result in the loss of some structural information. Additionally, results depend on the viability of the cells, sample preparation, calibration of the instrument, and quality of the reagents and qualified professional.

A third technique used for detecting biomarkers of interest is electrochemiluminescence (ECL), also known as electro generated chemiluminescence. ECL is the light emission process in which the species generated on an electrode surface undergoes an exergonic electron transfer reaction to form excited states that emit light. In this way, ECL detection consists of monitoring the production of photons and, thus, the intensity of the light produced during the electrochemical reaction in solution. This process combines electrochemical and spectroscopic methods, which improves the sensitivity and specificity of the method. However, the luminol reagent important for ECL provides a weak signal and low solubility in neutral aqueous solution. Another disadvantage of this method is the frequent encrustation of the electrodes.

Thus, there is a need to develop new diagnostic methods and devices capable of determining the presence and profile of biomarkers that may be associated with a particular disorder or disease.

### SUMMARY OF THE DISCLOSURE

The present disclosure includes methods, devices, and systems to measure biomarkers associated with a particular disorder or disease. The disclosed methods, devices and systems may be embodied in a variety of ways.

In certain embodiments, the disclosed methods, devices and systems may be used to detect biomarkers that are characteristic of any disorder or disease. In some cases, the disease may result from infection by a pathogen. The method to detect the presence of a biomarker in a subject may include the steps of: (a) obtaining a sample from the individual; (b) applying the sample to a modified biosensor, the biosensor comprising: (i) a glutaraldehyde-functionalized carbon nanoparticle paste; (ii) one or more immobilized antibodies specific to the biomarker; and (iii) optionally, a blocking agent; (c) applying an alternating voltage to the modified biosensor; (d) measuring an electrical impedance spectroscopy (EIS) signal to determine the presence of the biomarker. In an embodiment, a blocking agent is not required as the sample itself (e.g., plasma, blood, a nasal swab or saliva) serves as a blocking agent.

In certain embodiments, disclosed is a method to detect antigens specific to COVID-19 in a subject. The method may comprise the steps of: (a) obtaining a sample from the individual; (b) applying the sample to a modified biosensor, the biosensor comprising: (i) a glutaraldehyde-functionalized carbon nanoparticle paste; (ii) one or more immobilized antibodies specific to the biomarker; and (iii) optionally a blocking agent; (c) applying an alternating voltage to the biosensor; and (d) measuring an electrical impedance spectroscopy (EIS) signal to determine the presence of an antigen specific to COVID-19.

In an embodiment, the antigen is an antigen specific to COVID-19, e.g., the Sars-Co-2 S protein. Additionally and/or alternatively, the method may comprise detecting the presence of a modified COVID-19 specific antigen (e.g. Sars-CoV-2 S protein) in samples from patients in remission for COVID-19. In certain embodiments, the EIS signal for a subject who is in remission for COVID-19 can be distinguished from an EIS signal for a subject who has an active COVID-19 infection. Thus, in certain embodiments, the EIS signals for (i) a subject who is actively infected with COVID-19; or (ii) is in remission from a COVID-10 infection; or (iii) has never been exposed to COVID-19 have defined characteristics (i.e., shape and intensity) can be distinguished. The modified antigen may comprise a fragment of the antigen, or an antigen that has been biologically modified in some other manner. The samples from patients in remission may still test positive for COVID-19 antibodies but are negative for COVID-19 virus (e.g., as measured by PCR or other methods). As such, the assay may provide a fingerprint that is characteristic of subjects who have been exposed to COVID-19 but who are no longer infected. A variety of biological samples may be used. For example, in some embodiments, the sample may be one of a nasal swab, blood, serum, plasma, peritoneal fluid, pleural fluid, cerebrospinal fluid, uterine fluid, saliva, or mucus. Or other biological samples may be used.

In another aspect, disclosed herein is a method of making a biosensor comprising: modifying a biosensor to comprise: (i) a glutaraldehyde-functionalized carbon nanoparticle paste; (ii) one or more immobilized antibodies; and (iii) optionally a blocking agent.

Also disclosed are devices and systems for measuring biomarkers of interest. For example, disclosed is a biosensor device comprising: (a) a first layer comprising a carbon nanoparticle; (b) a second layer comprising glutaraldehyde; and (c) a third layer comprising one or more immobilized antibodies. In some embodiments, the biosensor is formulated as a strip. In some cases, the device may be used as part of a system. Thus, in some embodiments is a system to detect the presence of a biomarker in an individual comprising: (a) the biosensor device of any preceding or subsequent illustration; (b) a power supply; (c) a computer or other electronic analysis device; (d) an interface; and (e) a storage device.

In another aspect, disclosed herein is a computer-program product tangibly embodied in a non-transitory machine-readable storage medium, including instructions configured to perform any of the steps of the methods disclosed herein or run any part of a device disclosed herein or run any part of a system disclosed herein.

### BRIEF DESCRIPTION OF THE FIGURES

FIG. 1 depicts a linking mechanism between the aldehyde functional group and antibody amino group, where R represents the continuation of the glutaraldehyde carbon chain and Z represents the continuation of the peptide or amino acid carbon chain in accordance with an embodiment of the disclosure.

FIG. 2 shows a Nyquist diagram for a carbon screen-printed electrode (SPE) in accordance with an embodiment of the disclosure.

FIG. 3 shows a Nyquist diagram for Layer 2 (carbon nanoparticles paste functionalized with glutaraldehyde) of the SPE in accordance with an embodiment of the disclosure.

FIG. 4 shows a Nyquist diagram for Layer 3 (carbon nanoparticle paste functionalized with glutaraldehyde and immobilized antibody) of the SPE in accordance with an embodiment of the disclosure.

FIG. 5 shows a Nyquist diagram for Layer 4 (carbon nanoparticle paste functionalized with glutaraldehyde, immobilized antibody, and blocking agent) of the SPE in accordance with an embodiment of the disclosure.

FIG. 6 shows the Nyquist diagram for Layer 5 (carbon nanoparticle paste functionalized with glutaraldehyde, immobilized antibody, blocking agent, and antigen-antibody complex) of the SPE in accordance with an embodiment of the disclosure.

FIG. 7 shows variation of resistance along the formation of the self-assembled layers of the biosensor in accordance with an embodiment of the disclosure. In FIG. 7, G corresponds to carbon nanoparticles paste functionalized with glutaraldehyde, A corresponds to antibody immobilization, B corresponds to non-specific binding block and C corresponds to antigen-antibody reaction.

FIG. 8 shows the impedance levels corresponding to cytokine concentration in accordance with an embodiment of the disclosure.

FIG. 9 shows the standard curve for the charge transfer resistance (RCT) of TNF-α concentrations in accordance with an embodiment of the disclosure.

FIG. 10 shows the standard curve for the charge transfer resistance (RCT) of IL-1 concentrations in accordance with an embodiment of the disclosure.

FIG. 11 shows two standard curves for the charge transfer resistance (RCT) of IL-6 concentrations in accordance with an embodiment of the disclosure. FIG. 11A provides a standard curve at different concentrations of IL-6 (10, 20, 40, and 80 pg/mL). FIG. 11B provides a standard curve at different concentrations of IL-6 (100, 250, 500, and 750 pg/mL).

FIG. 12 shows the Nyquist impedance diagrams of carbon electrodes modified by self-assembled monolayer (SAM) with IL-1β monoclonal antibody, coupled after reaction with serum+cytokine, with a concentration of 50 pg·mL-1 for TNF-α, 10 pg·mL-1 for IL-6 and 5 pg·mL-1 for IL-1β in accordance with an embodiment of the disclosure.

FIG. 13 shows the Nyquist impedance diagrams of carbon electrodes modified by SAM with monoclonal antibody anti-TNF-α, coupled after reaction with serum+cytokine, with a concentration of 50 pg·mL-1 for TNF-α, 10 pg·mL-1 for IL-6 and 5 pg·mL-1 for IL-1β in accordance with an embodiment of the disclosure.

FIG. 14 shows an exemplary SPE comprising a base, first, second, third, and fourth layer of a that can be modified in accordance with an embodiment of the disclosure.

FIG. 15 depicts as system in accordance with an embodiment of the disclosure.

FIG. 16 depicts an embedded system hardware scheme that uses a sine wave signal generated by AD5933 in accordance with an embodiment of the disclosure.

FIG. 17 depicts a hardware block diagram in accordance with an embodiment of the disclosure.

FIG. 18 depicts a screen-printed electrode (SPE) in accordance with an embodiment of the disclosure.

FIG. 19 depicts the surface modification reaction to immobilize IL-1β, TNF-α, and IL-6 antibodies on the surface of the SPE in accordance with an embodiment of the disclosure.

FIG. 20 depicts a reduction oxidation reaction in accordance with an embodiment of the invention.

FIG. 21 depicts the antigen profile from a sample from a subject who is negative for COVID-19 (left panel), positive for COVID-19 (middle panel) or in remission from a prior infection with COVID-19) in accordance with an embodiment of the disclosure.

### DETAILED DESCRIPTION OF THE DISCLOSURE

Disclosed herein are devices, methods, and systems that demonstrate surprising speed and sensitivity for detecting biomarkers. Detection can be achieved in a shorter timeframe than with currently available methods. The present disclosure describes the use of modified electrodes for the detection of biomarkers.

The ensuing description provides exemplary embodiments only, and is not intended to limit the scope, applicability or configuration of the disclosure. Rather, the ensuing description of the exemplary embodiments will provide those skilled in the art with an enabling description for implementing various embodiments. It is understood that various changes may be made in the function and arrangement of elements without departing from the scope of the invention as set forth herein.

Specific details are given in the following description to provide a thorough understanding of the embodiments. However, it will be understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

### Definitions

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, microbiology, genetics and protein and nucleic acid chemistry and hybridization described herein are those well-known and commonly used in the art. Known methods and techniques are generally performed according to conventional methods well known in the art and as described in various general and more specific references that are discussed throughout the present specification unless otherwise indicated. Enzymatic reactions and purification techniques are performed according to manufacturer's specifications, as commonly accomplished in the art or as described herein. The nomenclatures used in connection with the laboratory procedures and techniques described herein are those well-known and commonly used in the art.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more, e.g. 1 to 6.1, and ending with a maximum value of 10 or less, e.g., 5.5 to 10. Additionally, any reference referred to as being “incorporated herein” is to be understood as being incorporated in its entirety.

The following terms, unless otherwise indicated, shall be understood to have the following meanings:

When introducing elements of the present disclosure or the embodiment(s) thereof, the articles “a”, “an”, “the” and “said” are intended to mean that there are one or more of the elements. The terms “comprising”, “including” and “having” are intended to be inclusive and mean that there may be additional elements other than the listed elements. It is understood that aspects and embodiments of the disclosure described herein include “consisting” and/or “consisting essentially of” aspects and embodiments.

The term “and/or” when used in a list of two or more items, means that any one of the listed items can be employed by itself or in combination with any one or more of the listed items. For example, the expression “A and/or B” is intended to mean either or both of A and B, i.e. A alone, B alone or A and B in combination. The expression “A, B and/or C” is intended to mean A alone, B alone, C alone, A and B in combination, A and C in combination, B and C in combination or A, B, and C in combination.

Various aspects of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used herein, the term “immunoglobulin” or “antibody” refers to a polypeptide consisting of one or more polypeptides substantially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are typically classified as either kappa or lambda. Heavy chains are typically classified as gamma, mu, alpha, delta, or epsilon, which in turn define the immunoglobulin classes, IgG, IgM, IgA, IgD and IgE, respectively. A typical immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms “variable light chain” (VL) and “variable heavy chain” (VH) refer to these light and heavy chains respectively. An antibody can be specific for a particular antigen. The antibody or its antigen can be either an analyte or a binding partner. Antibodies exist as intact immunoglobulins or as a number of well-characterized fragments produced by digestion with various peptidases. Thus, for example, pepsin digests an antibody below the disulfide linkages in the hinge region to produce F(ab)′2, a dimer of Fab which itself is a light chain joined to VH-CH1 by a disulfide bond. The F(ab)′2 may be reduced under mild conditions to break the disulfide linkage in the hinge region thereby converting the (Fab′)2 dimer into an Fab′ monomer. The Fab′ monomer is essentially an Fab with part of the hinge region. While various antibody fragments are defined in terms of the digestion of an intact antibody, one of ordinary skill in the art will appreciate that such Fab′ fragments may be synthesized de novo either chemically or by utilizing recombinant DNA methodology. Thus, the term “antibody,” as used herein also includes antibody fragments either produced by the modification of whole antibodies or synthesized de novo using recombinant DNA methodologies. In some embodiments, antibodies are single chain antibodies, such as single chain Fv (scFv) antibodies in which a variable heavy and a variable light chain are joined together (directly or through a peptide linker) to form a continuous polypeptide. A single chain Fv (“scFv”) polypeptide is a covalently linked VH:VL heterodimer which may be expressed from a nucleic acid including VH- and VL-encoding sequences either joined directly or joined by a peptide-encoding linker. (See, e.g., Huston, et al. (1988) Proc. Nat. Acad. Sci. USA, 85:5879-5883) A number of structures exist for converting the naturally aggregated, but chemically separated light and heavy polypeptide chains from an antibody V region into an scFv molecule which will fold into a three dimensional structure substantially similar to the structure of an antigen-binding site. See, e.g. U.S. Pat. Nos. 5,091,513 and 5,132,405 and 4,956,778.

As used herein, the phrase “specifically binds,” when used in the context of describing a binding relationship of a particular molecule to a protein or peptide, refers to a binding reaction that is determinative of the presence of the protein in a heterogeneous population of proteins and other biologics. Thus, under designated binding assay conditions, the specified binding agent (e.g., an antibody) binds to a particular protein at least two times the background and does not substantially bind in a significant amount to other proteins present in the sample. Specific binding of an antibody under such conditions may require an antibody that is selected for its specificity for a particular protein or a protein but not its similar “sister” proteins. A variety of immunoassay formats may be used to select antibodies specifically immunoreactive with a particular protein or in a particular form. For example, solid-phase ELISA immunoassays are routinely used to select antibodies specifically immunoreactive with a protein (see, e.g., Harlow & Lane, Antibodies, A Laboratory Manual (1988) for a description of immunoassay formats and conditions that can be used to determine specific immunoreactivity). Typically a specific or selective binding reaction will be at least twice background signal or noise and more typically more than 10 to 100 times background. On the other hand, the term “specifically bind” when used in the context of referring to a polynucleotide sequence forming a double-stranded complex with another polynucleotide sequence describes “polynucleotide hybridization” based on the Watson-Crick base-pairing, as provided in the definition for the term “polynucleotide hybridization method.”

As used herein, an “increase” or a “decrease” refers to a detectable positive or negative change in quantity from a comparison control, e.g., an established standard control. An increase is a positive change that is typically at least 10%, or at least 20%, or 50%, or 100%, and can be as high as at least 2-fold or at least 5-fold or even 10-fold of the control value. Similarly, a decrease is a negative change that is typically at least 10%, or at least 20%, 30%, or 50%, or even as high as at least 80% or 90% of the control value. Other terms indicating quantitative changes or differences from a comparative basis, such as “more,” “less,” “higher,” and “lower,” are used in this application in the same fashion as described above. In contrast, the term “substantially the same” or “substantially lack of change” indicates little to no change in quantity from the standard control value, typically within 10% of the standard control, or within +5%, 2%, or even less variation from the standard control.

As used herein, the term “biomarker” or a “biomarker of interest” is any biomolecule that may provide biological information about the physiological state of an organism. In certain embodiments, the presence or absence of the biomarker may be informative. In other embodiments, the level of the biomarker may be informative. In an embodiment, the biomarker of interest may comprise a peptide, a hormone, a nucleic acid, a lipid or a protein. Or, other biomarkers may be measured.

As used herein, the term “body fluid” or “sample” or “biological sample” refers to a sample obtained from a biological source, including, but not limited to, an animal, a cell culture, an organ culture, and the like. Suitable samples include nasal swabs, blood, plasma, saliva, serum, urine, saliva, tear, cerebrospinal fluid, or other liquid aspirate.

As used herein, the terms “individual” and “subject” are used interchangeably. A subject may comprise an animal. Thus, in some embodiments, the biological sample is obtained from a mammalian animal, including, but not limited to a dog, a cat, a horse, a rat, a monkey, and the like. In some embodiments, the biological sample is obtained from a human subject. In some embodiments, the subject is a patient, that is, a living person presenting themselves in a clinical setting for diagnosis, prognosis, or treatment of a disease or condition.

As used herein, the terms “biosensor” and “biosensor device” are used interchangeably. A biosensor is an analytical device which converts a biological responses into a quantifiable and processable signal. In some embodiments, the biosensor may be formulated as a strip.

### Biosensor Device

In certain embodiments, disclosed is a biosensor device. For example, in certain embodiments, a biosensor device may comprise: (a) a first layer comprising a carbon nanoparticle; (b) a second layer comprising glutaraldehyde; and (c) a third layer comprising one or more immobilized antibodies. In certain embodiments, the device comprises a carbon paste electrode. In some instances, the carbon paste electrode comprises multiple layers. In further embodiments, the carbon paste electrode comprises at least one of carbon, mineral oil, and/or glutaraldehyde. In some embodiments, one or more layers are passivated. For example, skim milk may be used to passivate an electrode. In some embodiments, the biosensor is formulated as a strip.

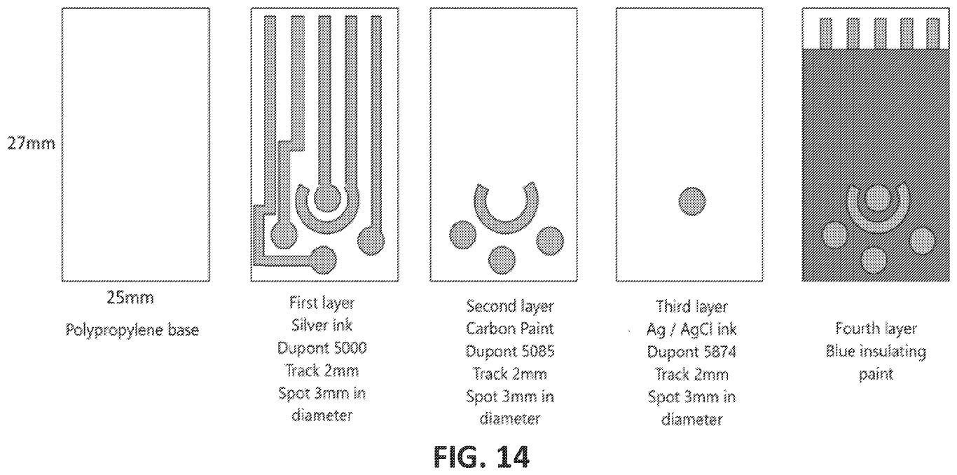

In some embodiments, the biosensor device is a modified screen printed electrode (SPE) as shown in FIG. 14. For example, a SPE may comprise a polypropylene base layer, a first layer comprising silver ink, a second layer comprising carbon paint, a third layer comprising Ag/AgCl (silver/silver chloride) ink and fourth layer comprising an insulating paint. In some embodiments, the SPE is modified to produce a biosensor device comprising: (a) a first layer comprising a carbon nanoparticle; (b) a second layer comprising glutaraldehyde; and (c) a third layer comprising one or more immobilized antibodies. For example, and as disclosed herein the SPE may be physically modified by insertion of carbon nanoparticles (Vulcan XC 75) and mineral oil and functionalized with glutaraldehyde.

Thus, in one aspect of the present disclosure, the method of making a biosensor device comprising modifying a biosensor. In some embodiments, the method of making a biosensor comprises modifying a screen printed electrode (SPE) to comprise: (i) a glutaraldehyde-functionalized carbon nanoparticle paste; (ii) one or more immobilized antibodies; and (iii) optionally a blocking agent. In some embodiments, the biosensor is a screen-printed electrode (SPE). SPEs allow for the mass production of inexpensive electrochemical biosensors. SPEs can be configured in a variety of ways with varying compositions. For example, the electrode size and thickness, ink composition, materials, and size can all be varied. In some embodiments, the SPE is modified by inserting functionalized carbon nanoparticles into the device. In some instances the carbon nanoparticles are functionalized with glutaraldehyde. For example, mineral oil, glutaraldehyde solution, and carbon nanoparticles may be mixed together to form a glutaraldehyde-functionalized carbon nanoparticle paste. In some embodiments, the blocking agent comprises an external blocking agent such as milk or BSA.

### Antibodies to Biomarkers

In some embodiments, an antibody specific to the biomarker of interest is immobilized on the surface of the biosensor. Carbon nanoparticle paste can be functionalized with glutaraldehyde to allow for the antibody to be immobilized on the surface. Following antibody immobilization, in some embodiments, the blocking agent is used to prevent non-specific binding on the surface of the electrode. In some embodiments the blocking agent is milk or BSA. For example, skim milk may be used to passivate an electrode. In other embodiments, an external blocking agent is not required as the function of the blocking agent is provided by the sample from the subject. For example, bodily fluids, including but not limited to blood, mucus, and nasal swabs, may comprise a matrix that acts as a blocking agent.

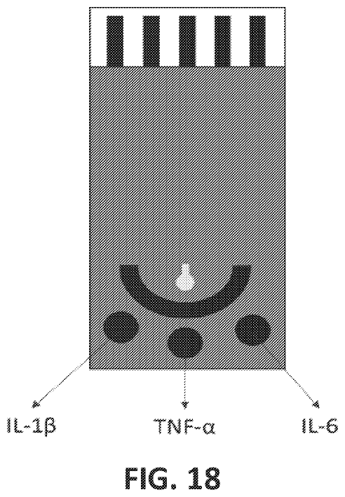

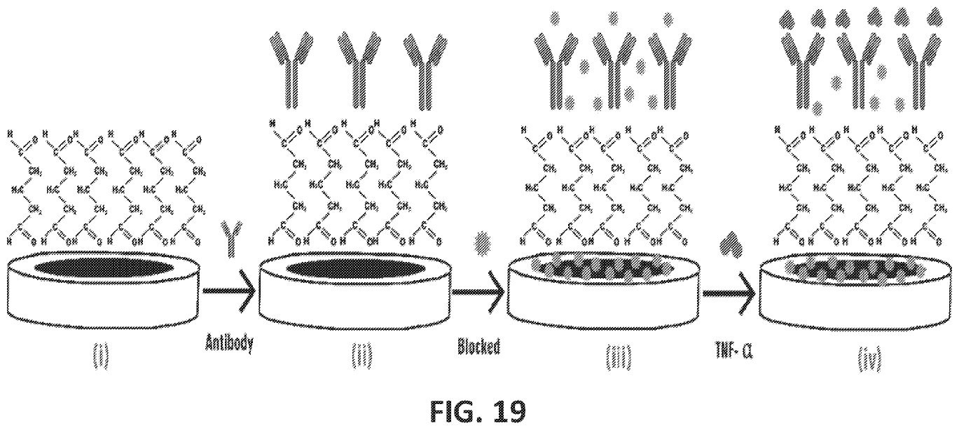

In some embodiments, the device further comprises a component for capturing a biomarker of interest. In some embodiments, the biosensor comprises antibodies specific for one or more biomarkers (FIG. 18). In certain instances, the device comprises a monoclonal antibody (FIG. 19). Monoclonal antibodies can be used to immobilize biomarkers. In some embodiments, the biosensor comprises at least 1, 2, 3, 5, 10, 15, 20, or 25 types of antibodies.

### Detection of Biomarkers

In certain embodiments, the method to detect the presence of a biomarker in a subject may include: (a) obtaining a sample from the individual and applying it to the modified biosensor; (b) applying the same to a biosensor, the biosensor comprising: (i) a glutaraldehyde-functionalized carbon nanoparticle paste; (ii) one or more immobilized antibodies specific to the biomarker; and (iii) optionally a blocking agent; (c) applying an alternating voltage to the modified biosensor; (d) measuring an electrical impedance spectroscopy (EIS) signal to determine the presence of the biomarker. In an embodiment, a blocking agent is not required as the sample itself (e.g., plasma, blood, a nasal swab or saliva) serves as a blocking agent. In some embodiments, the biosensor is formulated as a strip

As depicted in FIG. 19, in some embodiments, the method comprises (i) modifying a biosensor such that (ii) an antibody specific to a biomarker of interest may be immobilized on the surface of the biosensor, (iii) exposing the biosensor to a blocking agent such that non-specific binding is reduced, and (iv) exposing the biosensor to sample comprising the biomarker of interest (e.g., TNF-α) such that the immobilized antibody captures the biomarker of interest.

Electrochemical impedance spectroscopy (EIS) is a technique for measuring the electrical resistance (impedance) of the metal/solution interface over a wide range of frequencies. Electrochemical impedance can be measured by applying an alternating current (AC) potential to an electrochemical cell and then measuring the current through the cell.

In certain embodiments, the device is an immunosensor for detection of a biomarker using electrochemical impedance spectroscopy (EIS). In some embodiments, the biomarker is associated with an active inflammatory response. For example, Tumor Necrosis Factor-α (TNF-α) is a cytokine that plays an important role in activating inflammatory and immune responses. Abnormal levels of this cytokine are associated with diseases such as rheumatoid arthritis (RA), systemic lupus erythematosus and Crohn's disease. Quantification of TNF-α in human serum in clinical and research laboratories is based on immunoenzymatic techniques such as ELISA. In some embodiments, the biomarker is associated with a viral antigen specific for a viral infection. For example, the biomarker may be the Sars-CoV2 spike (S) protein, which is specific for COVID-19. In some embodiments, the biomarker is associated with a bacterial antigen specific for a bacterial infection.

In one embodiment, disclosed is a method to detect antigens specific to COVID-19 in a subject. A method to detect antigens specific to COVID-19 in a subject comprising: (a) obtaining a sample from the individual and applying it to the modified biosensor; (b) applying the same to a biosensor, the biosensor comprising: (i) a glutaraldehyde-functionalized carbon nanoparticle paste; (ii) one or more immobilized antibodies specific to the biomarker; and (iii) optionally a blocking agent; (c) applying an alternating voltage to the modified biosensor; and (d) measuring an electrical impedance spectroscopy (EIS) signal to determine the presence of an antigen specific to COVID-19.

In an embodiment, the antigen is an antigen specific to COVID-19, e.g., the Sars-CoV-2 S protein. Additionally and/or alternatively, the method may comprise detecting the presence of a modified COVID-19 specific antigen (e.g. Sars-CoV-2 S protein) in samples from patients in remission for COVID-19. The samples from patients in remission may still test positive for COVID-19 antibodies but are negative for COVID-19 virus (e.g., as measured by PCR or other methods). As such, the assay may provide a fingerprint that is characteristic of subjects who have been exposed to COVID-19 but who are no longer infected.

The modified antigen may comprise a fragment of the antigen, or an antigen that has been biologically modified in some other manner. For example, the modified Sars-CoV2 antigen may be the result of biochemical modifications (e.g., glycosylation, alkylation, etc.) or the modified Sars-CoV2 antigen may be a degraded portion of the Sars-CoV2 antigen. In some embodiments, the modified Sars-CoV2 antigen is a modified Sars-CoV2 S protein. In some embodiments, the methods and devices described herein may be used to monitor biomarkers in blood samples from patients with autoimmune disease. In other embodiments, viral infections may be detected.

In some embodiments the sample is a bodily fluid. Examples of bodily fluids include but are not limited to blood, serum, plasma, peritoneal fluid, pleural fluid, cerebrospinal fluid, uterine fluid, saliva, and mucus.

In some embodiments, the method comprises determining a biomarker profile. In some instances, the biomarker profile determines whether subject suffers from a viral infection, bacterial infection, or an inflammatory condition. The viral infection is at least one of influenza virus, herpes simplex virus (HSV), human immunodeficiency virus (HIV) type 1 (HIV-1), HIV-2 Group A, HIV-2 Group B, HIV-1 Group M, Hepatitis B, Hepatitis Delta, Ebola virus, Marburg virus, Cueva virus, West Nile Virus, Epstein-Barr Virus, Dengue Virus, adenovirus B, adenovirus C, adenovirus E, Virus, Parainfluenza Virus type 1, Parainfluenza Virus type 2, Parainfluenza Virus, Coronavirus, 229E, Coronavirus HKU1, Coronavirus OC43, Coronavirus NL63, SARS-CoV, MERS-CoV, or SARS-CoV-2.

For example, and as disclosed in detail herein, in certain embodiments, the biomarker or biomarker profile may comprise an antigen specific to COVID-19, as for example, but not limited to the Sars-CoV-2 S protein. Also, the method may comprise detecting the presence of a modified COVID-19 specific antigen (e.g. Sars-CoV2 S protein). In some instances the modified COVID-19 specific antigen may be present in samples from patients in remission for COVID-19. The modified antigen may comprise a fragment of the antigen, or an antigen that has been biologically modified in some other manner. The samples from patients in remission may still test positive for COVID-19 antibodies but are negative for COVID-19 virus (e.g., as measured by PCR or other methods). As such, the assay may provide a fingerprint that is characteristic of subjects who have been exposed to COVID-19 but who are no longer infections.

In other embodiments, the inflammatory condition is at least one of rheumatoid arthritis, multiple sclerosis, myocardial infarction, COPD, chronic nephritis, chronic hepatitis, chronic pancreatitis, Type 2 diabetes, systemic lupus erythematosus (SLE), Alzheimer's disease, Parkinson's disease (PD), or inflammatory bowel disease (IBD).

In yet other embodiments, the bacterial infection may comprise an infection caused by at least one of Bordetella pertussis, Mycobacterium tuberculosis (MTB), Staphylococcus aureus, Methicillin-Resistant Staphylococcus aureus (MRSA), Group A streptococcus, Group B Streptococcus, Haemophilus parainfluenzae, or Klebsiella pneumoniae.

In some embodiments, the biomarker is a viral protein or a cytokine. In certain embodiments, the viral protein is the Sars-Cov2 Spike (S) protein, which is specific for a Sars-Cov2 infection. In further embodiments the cytokine is a pro-inflammatory cytokine. For example, pro-inflammatory cytokines include, but are not limited to tumor necrosis factor alpha (TNF-α), interleukin-1 beta (IL-1β), interleukin-6 (IL-6), interleukin-8 (IL-8), interleukin-10 (IL-10), interleukin-12 (IL-12), interferon gamma (IF-γ), CXCL10, MCP1, and MIP1α.

Tumor Necrosis Factor-α (TNF-α)

Tumor Necrosis Factor-α (TNF-α) plays a central role in the initiation and regulation of the cytokine cascade during an inflammatory response. It is produced as a 26 kDa membrane-bound precursor molecule that is cleaved by the TNF-α converting enzyme to produce a 17 kD active cytokine. In inflammatory states or in diseases, TNF-α, together with several other pro-inflammatory mediators and neurotoxic substances is produced in high concentrations. TNF-α is a potential biomarker for diseases such as multiple sclerosis, Parkinson's disease, rheumatoid arthritis and diabetic retinopathy. It is also involved in the induction of granulocyte and macrophage colony stimulating factor (GM-CSF), which is related to tumor growth and progression in several types of cancers.

Interleukin 6 (IL-6)

Interleukin 6 (IL-6) is a pro-inflammatory cytokine that can be secreted by many types of cells through appropriate stimulation during infection, inflammation or cancer. IL-6 can be important for regulating the responses of type B and T cells, as well as coordinating the activity of the innate and adaptive immune system. Under normal conditions, the circulating concentration of IL-6 in the blood range from 1 to 5 pg/mL. This can, however, can easily increase to ng/mL ranges in pathological situations. IL-6 can be strongly induced during most, if not all, inflammatory, infectious and cancerous processes. Also, in sepsis, IL-6 levels can reach mg per mL levels, and high amounts of this cytokine in the brain lead to neurodegeneration. IL-6 is a promising indicator in the treatment of several diseases such as rheumatoid arthritis, Castleman's disease, heart diseases, sepsis and more recently in COVID-19.

Interleukin 1 Beta (IL-1β)

Interleukin 1 beta (IL-1β) is a member of the IL-1 cytokine family, being rapidly generated and released by different types of immune and non-immune cells in response to inflammatory signals. Normal concentrations of IL-1β in biological fluids are less than 10 pg/mL. This biomarker may be upregulated in diseases such as colon, breast, oral and skin cancer. The overproduction of this cytokine is also observed in patients suffering from epilepsy, stroke, Alzheimer's disease and other neurological disorders, as well as in autoimmune diseases, in which it is one of the main responsible for the activation of these diseases. It is more recently correlated with type 2 diabetes, being induced by glucose itself.

### Systems

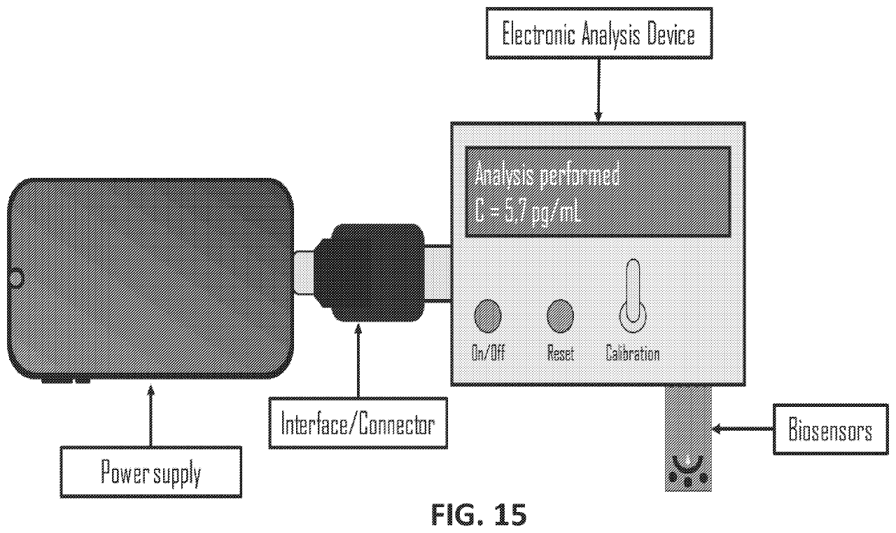

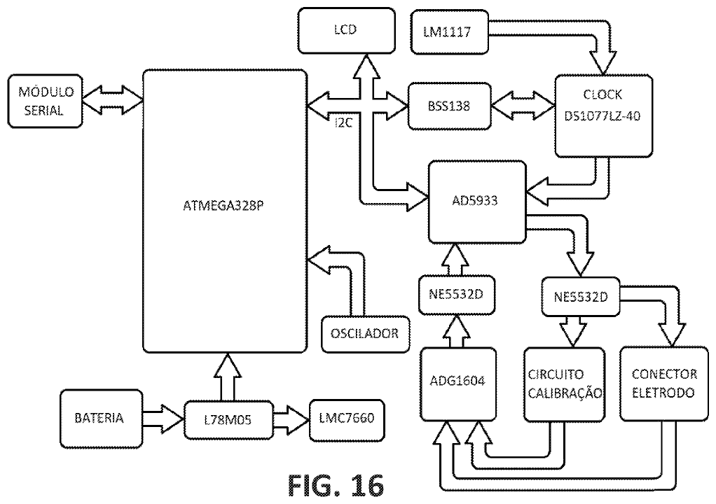

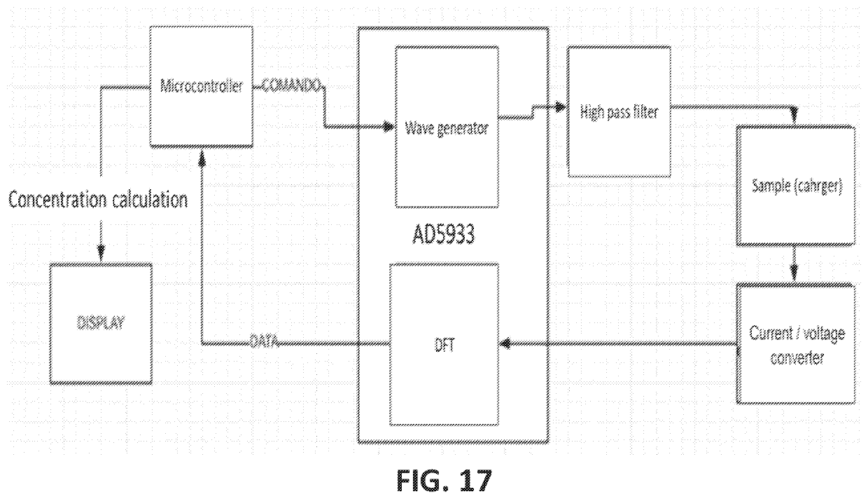

In some embodiments, the disclosure comprises systems (e.g., automated systems) comprising components for performing the methods disclosed herein. In some embodiments the system to detect the presence of a biomarker in an individual comprising: (a) the biosensor device of claim as described herein; (b) a power supply; (c) an electronic analysis device or computer; (d) an interface; and (e) a storage device (FIG. 15). In some embodiments, the biosensor is formulated as a strip. In some embodiments, the system further comprises a computer-program product tangibly embodied in a non-transitory machine-readable storage medium, including instructions configured to run any part of the system. In certain instances the system further comprises embedded system hardware comprising a high precision impedance converter system (e.g., AD5933) as depicted in FIGS. 16 and 17. In some embodiments, the embedded system uses a sine wave signal generated by AD5933 to perform electrochemical impedance spectroscopy (EIS) in accordance with an embodiment of the invention.

In some embodiments, the system further comprises hardware components for performing EIS (FIG. 17). In some instances, the system comprises: (i) a microcontroller (e.g. computer), (ii) an impedance converter system comprising (a) a wave generator and (b) a discrete Fourier transform (DFT), a high pass filter, a sample, a current/voltage converter, and a display. The wave generator allows an external complex impedance to be excited with a known frequency. The response signal from the impedance is sampled by the DFT and a DFT algorithm returns data at each output frequency. Thus, in some embodiments the microcontroller (e.g., computer) commands the impedance converter system (e.g., AD5933) to generate a sine wave. A high pass filter, which allows high frequencies to pass while blocking lower frequencies, may be used to provide a frequency cutoff. The sinusoidal current is applied to the sample to measure its impedance over a suitable frequency range. The current/voltage converter generates a response signal from the impedance and the impedance converter system DFT processes the signal, thereby generating data to the microprocessor to determine the concentration of the analyte which can then be displayed.

In another aspect, the system further comprises a computer-program product tangibly embodied in a non-transitory machine-readable storage medium, including instructions configured to perform any of the steps of the methods or run any part of the device of claims, or run any part of the system is embodied herein.

### EXAMPLES

The following examples have been included to provide guidance to one of ordinary skill in the art for practicing representative embodiments of the presently disclosed subject matter. In light of the present disclosure and the general level of skill in the art, those of skill can appreciate that the following examples are intended to be exemplary only and that numerous changes, modifications, and alterations can be employed without departing from the scope of the presently disclosed subject matter.

### Example 1. Formation of Carbon Nanoparticle Paste

Biosensors were developed using DROPSENS™ screen printed electrodes (SPE) and PSTrace5 software. Reference carbon electrodes DRP-110 were physically modified by insertion of carbon nanoparticles (Vulcan XC 75) and mineral oil and functionalized with glutaraldehyde. Mineral oil, a glutaraldehyde solution, and carbon nanoparticles were physically mixed to produce the carbon nanoparticle paste functionalized with glutaraldehyde. The paste was formed in a beaker or quartz surface by massaging 1.0 grams of Vulcan carbon (XC 72), subsequently adding 5.0% (v/v) glutaraldehyde solution until the volume of glutaraldehyde solution is completely absorbed, and subsequently adding 80 drops of mineral oil until a semi-solid, low-gloss paste was formed that easily detached from the beaker surface or the quartz pistil grade.

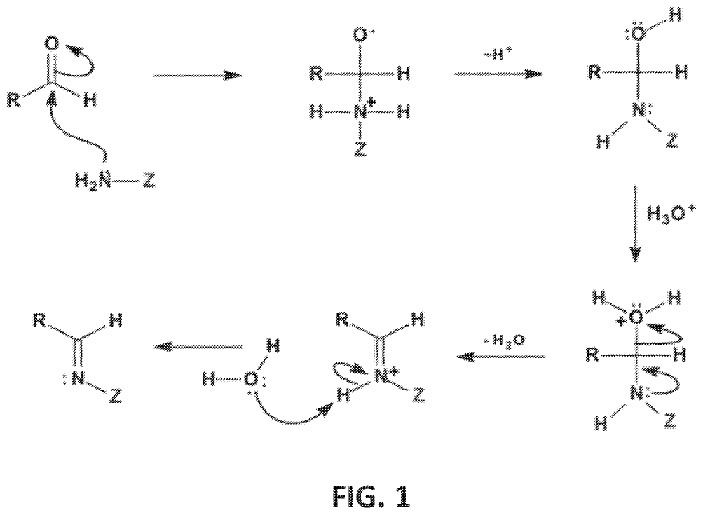

The ratio of glutaraldehyde solution, mineral oil, and carbon nanoparticles was optimized to for physical conformation and chemical capacity of the covalent bonds between the aldehyde group and amino groups present in the antibodies. Aldehyde groups on the surface of nanoparticles functionalized with glutaraldehyde react with amino groups of antibodies for covalent immobilization, forming an amide bond. Studies show that there are bonds with amides I and II present in the structures of the antibodies, since the antibodies are formed by peptides and, therefore, have amino acids in their structures which can react in different portions for the covalent bond with glutaraldehyde. FIG. 1 depicts the linking mechanism between the aldehyde functional group and antibody amino group, where R represents the continuation of the glutaraldehyde carbon chain and Z represents the continuation of the peptide or amino acid carbon chain.

### Example 2. Development of Biosensors with Carbon Nanoparticle Paste

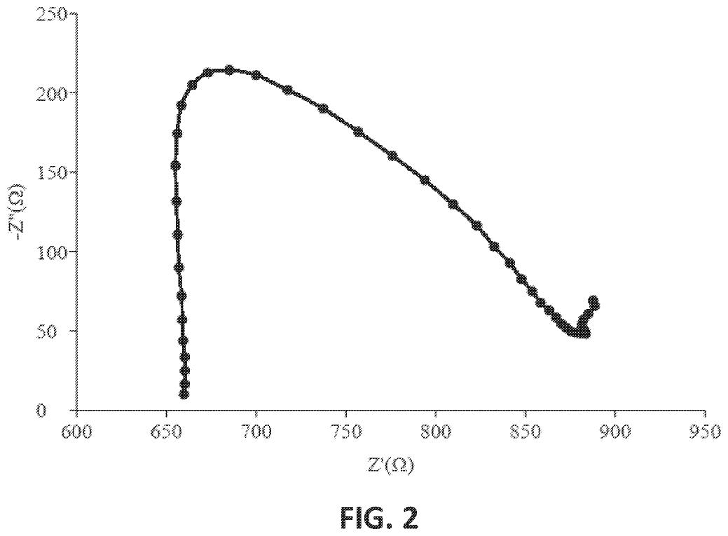

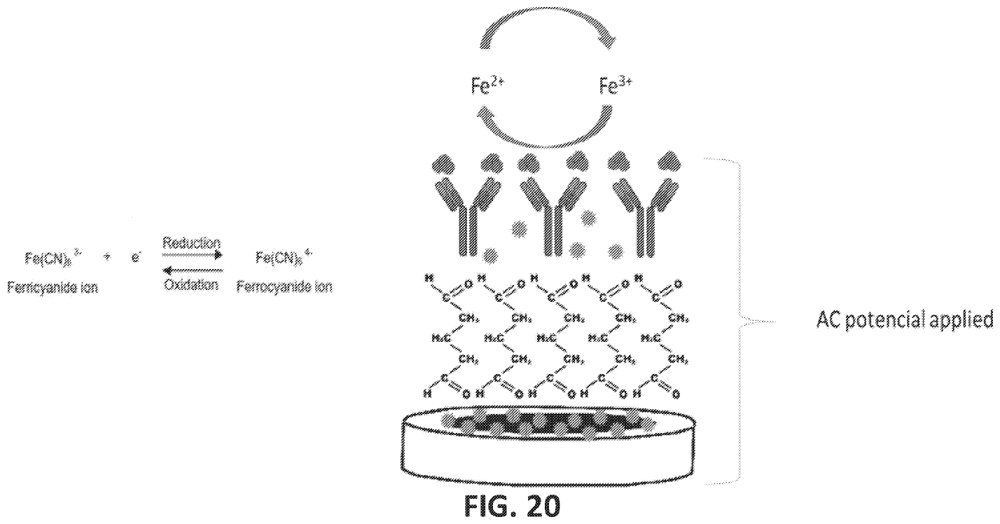

Before modifying the electrodes, the impedance of the SPE was quantified in order to observe the resistance of the solution to the electrode (Rs) and the amount of variation in the load transfer (Rct). Characterization analyses were performed for each layer of the biosensor using optimized parameters of analysis: 250 mV of amplitude variation and frequency variation from 10 kHz to 1 Hz, and using redox pair Potassium ferricyanide and potassium ferrocyanide (Fe+3/Fe+2) at the optimized concentration of 10 mM (FIG. 20). FIG. 2 shows the Nyquist diagram for a SPE analyzed by adding 50 μL of redox pair solution to the electrode surface. The Nyquist diagram (FIG. 2) was used to calculate the values of Rs, Rct, and Resistance to load transfer with correction and normalization Rctnor. The resistance value was checked at the highest frequency, which corresponds to the first point on the Nyquist graph (FIG. 2) and the Rct value was calculated. To calculate Rct, the equation of the circle was applied where Rct is equal to 2 times the highest real impedance value (Z′(Ω)), which corresponds to the highest phase angle value (−θ) or the highest imaginary impedance (−Z″(Ω)) reached before the capacitor, and the value of Rctnor calculated through the value of Rct minus the value of Rs.

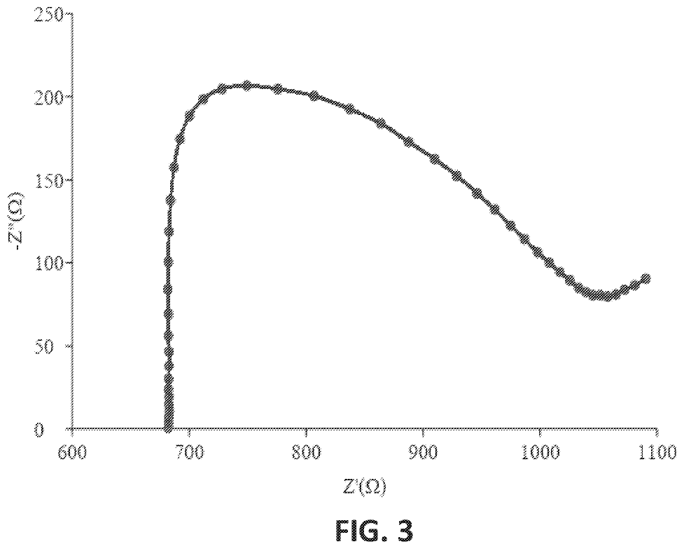

Following completion of the layer 1 analysis, the SPE surface was modified with the addition of carbon nanoparticle past functionalized with glutaraldehyde by applying between 5 and 7 mg of the paste to cover the entire reaction area. FIG. 3 shows the Nyquist diagram for Layer 2, after the addition of the carbon nanoparticles paste functionalized with glutaraldehyde, analyzed by adding 50 uL of redox solution to the electrode surface. The Nyquist diagram for layer 2 was used to verify the increase in resistance of the solution to the Rs electrode and the increase in Rct with a consequent increase in Rctnor when compared to the SPE. The observed effect is due to the physical and chemical properties of the paste comprising nanoparticulate systems functionalized with glutaraldehyde and having high porosity.

Next the immobilization process was performed. The ability of the developed biosensor to detect biomarkers is based on the antigen-antibody reaction process, which forms immunocomplexes with a certain amount of stability. Systems that use alternating current (AC) are preferable for these analyses.

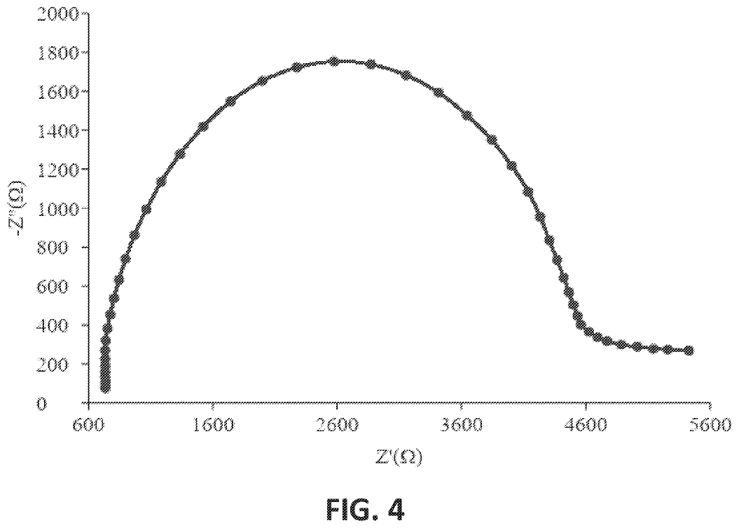

The immobilization process was performed by inserting 50 μL of antibody solution (A) with an optimized concentration of 1 mg·mL⁻¹, for 30 min and subsequently washing the electrode with 500 uL of PBS solution with tween 0.5% (m/v), thereby forming Layer 3. Layer 3 was analyzed by adding 50 uL of the redox pair solution to the electrode surface. FIG. 4 shows the Nyquist diagram for Layer 3 (after adding the carbon nanoparticle paste functionalized with glutaraldehyde and immobilizing the antibody). The Nyquist diagram for Layer 3 (FIG. 4) was used to verify the increase in the resistance of the solution to the Rs electrode and the large increase in Rct, with a consequent increase in Rctnor when compared to layer 2. The observed effect was due to the immobilization of antibodies on the electrode surface, which generates passivation and significant increase in system resistance.

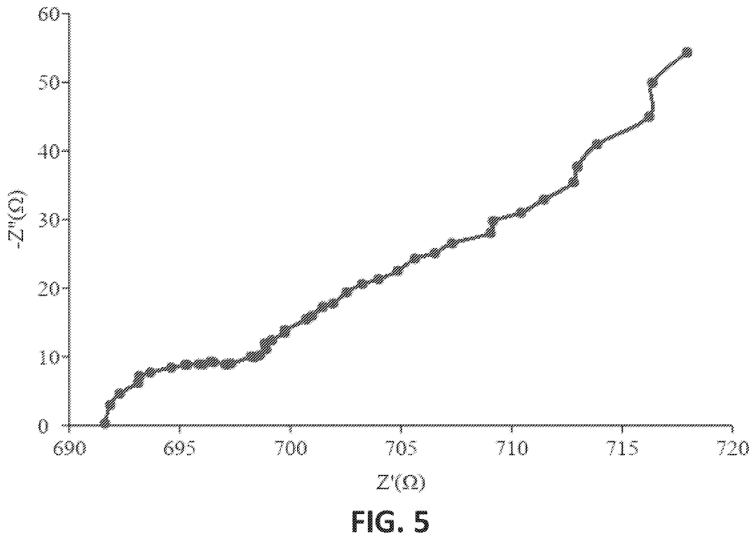

After immobilization, vacancies may be present on the sensor surface, which can generate non-specific responses to the system. In order to fill any present vacancies, 50 μL of blocking agent solution (B), such as BSA or skim milk, was inserted into the electrode at a concentration according to the antibody used for 10 min. Then, the electrode was washed with 500 μL of PBS solution with TWEEN 0.5% (m/v) and, analyzed by adding 50 uL of redox pair solution to the electrode surface. FIG. 5 shows the Nyquist diagram for Layer 4 (after adding the carbon nanoparticles paste functionalized with glutaraldehyde, immobilizing the antibody, and blocking nonspecific binding). The Nyquist Diagram for layer 4 (FIG. 5) was used to verify that there was a small decrease in the resistance of the solution to the Rs electrode and the diffusion phenomenon without formation of Rct, known as the Warbug (W) diffusion phenomenon. Thus, due to the Warburg phenomenon, the System Rctnor becomes 0, since Rct is zero resulting in a negative Rctnor. The observed effect is due to high passivation on the electrode surface which generates connections in the vacancies on the electrode surface that prevent redox processes from occurring easily. After the antibodies were immobilized and possible sensor surface vacancies were filled, the biosensor was ready for use.

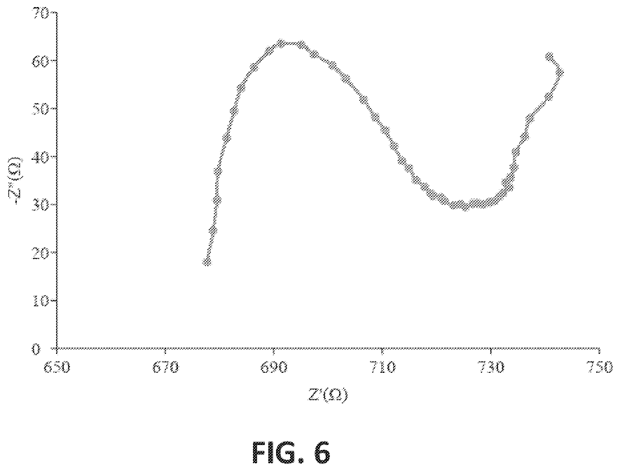

In order to test detection ability of the biosensor, 50 μL of antigen solution (IL-6, TNF-α, or IL-1β) was inserted at a concentration of 100 pg·mL-1, for 5 min into the electrode with 500 uL of PBS solution with 0.5% tween (w/v) and analyzed by adding 50 uL of redox pair solution to the electrode surface. FIG. 6 shows the Nyquist diagram for Layer 5, after adding the carbon nanoparticles paste functionalized with glutaraldehyde, immobilizing the antibody, blocking nonspecific binding and antigen-antibody reaction. FIG. 6 was used to verify the decrease in the resistance of the solution to the Rs electrode and the formation of Rct, in addition, Warburg phenomena (W) was still observed after 710Ω. The observed effect was due to the antigen-antibody interaction which generates stable structures sufficient for the observation of resistive phenomena arising from redox reactions of Fe+3/Fe+2.

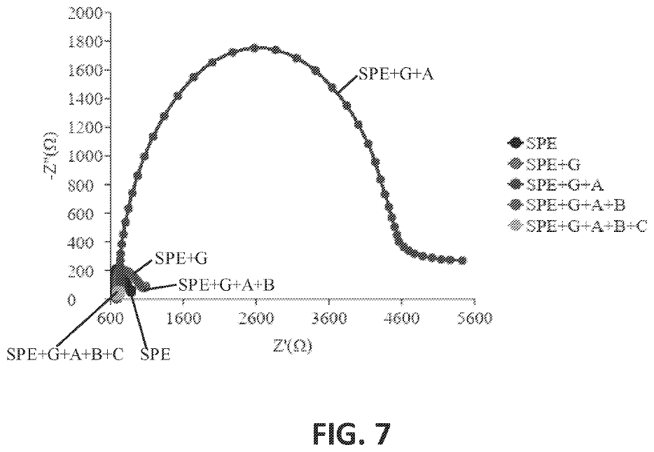

FIG. 7 shows variation of resistance along the formation of the self-assembled layers of the biosensor. G corresponds to carbon nanoparticles paste functionalized with glutaraldehyde, A corresponds to antibody immobilization, B corresponds to non-specific binding block and C corresponds to antigen-antibody reaction. The results indicated that the antibodies were fixed on the electrode surface and the system responded to the presence of the cytokine. The same behavior was observed for all other tested cytokines. Table 1 shows the Rs, Rct, and Rctnor values for each layer.

TABLE 1
Rs, Rct, and Rctnor data for biosensor layers

| Layer | Rs(Ω) | Rct(Ω) | Rctnor(Ω) |
|---|---|---|---|
| SPE | 659.8 | 1346.2 | 686.4 |
| SPE + G | 682.1 | 1456.1 | 774.0 |
| SPE + G + A | 735.5 | 2370.6 | 1635.1 |
| SPE + G + A + B | 691.6 | 0.0 | 0.0 |
| SPE + G + A + B + C | 677.7 | 1382.7 | 705.0 |

The data (Table 1) demonstrated the variation of the reaction systems on the electrode surface, which generated Randles circuit. Randles circuits are well known in systems using alternating current electrochemical biosensors.

### Example 3. Development of Standard Curves

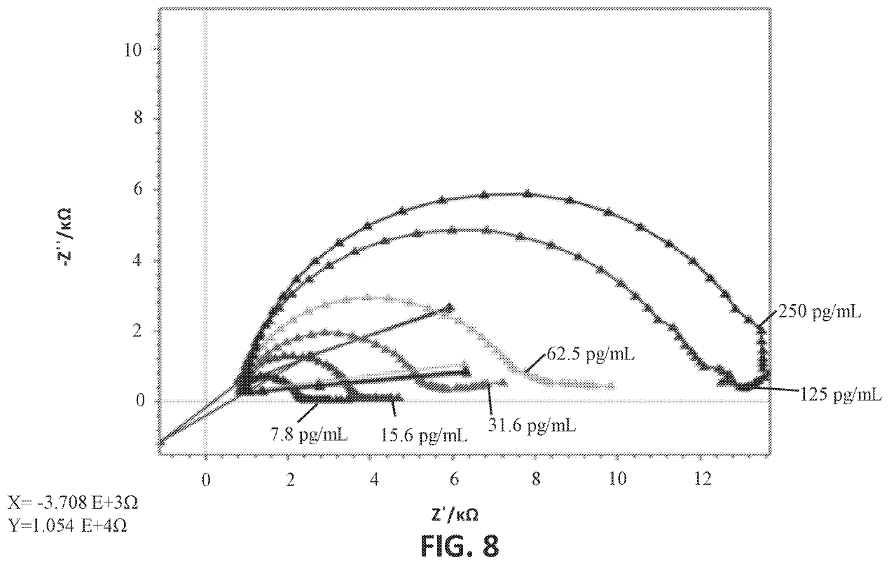

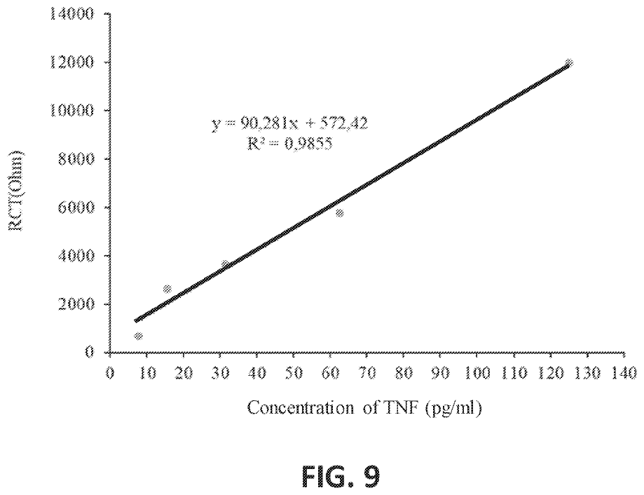

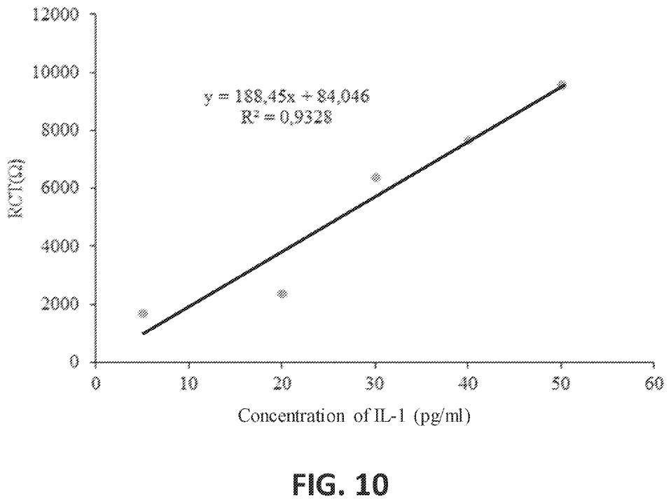

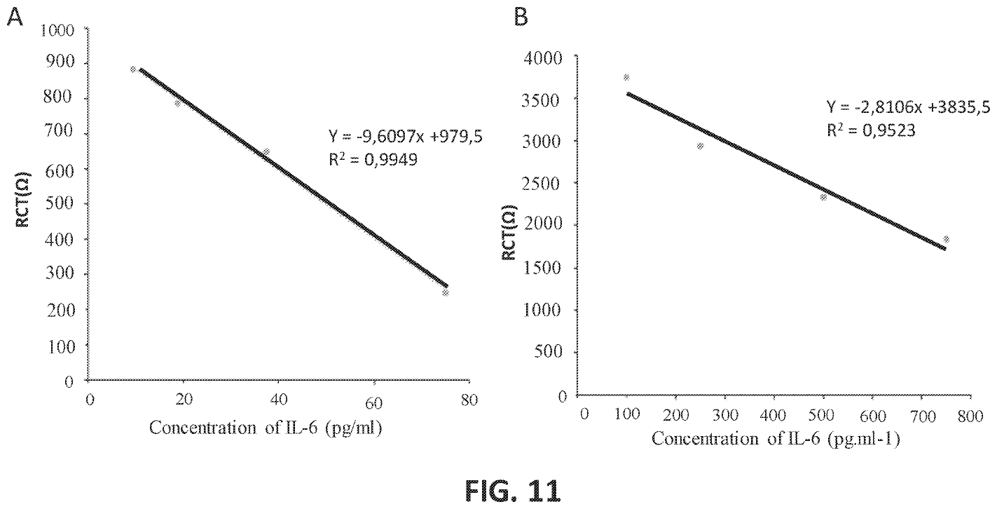

In order to construct a standard curve from which a mathematical equation to be used in the equipment software, six printed carbon electrodes were modified and impedance spectroscopy measurements of increasing cytokine concentrations were made. Anti-TNF-α (1 mg/mL) was inserted into the electrode for 30 min to immobilize the antibodies. Then the electrode was washed with a pH 7.4 buffer solution. TNF-α cytokine was added to each electrode for 30 minutes at concentrations of 250, 125, 62.5, 31.3, 15.6 and 7.8 pg/mL. The electrode was washed with a pH 7.4 buffer solution. The electrode was analyzed with a redox probe and the impedance spectrum of the standard curve solutions was determined. The results followed an expected linearity, with the lowest impedance levels corresponding to the lowest levels of cytokine concentration and the highest impedance levels corresponding to the highest levels of cytokine concentration (FIG. 8). FIG. 9 shows the standard curve for TNF-α. The same methods for standard curve development were completed for IL-6 and IL-1β. FIG. 11 shows the standard curve for IL-6 and FIG. 10 shows the standard curve for IL-1β.

### Example 4. Biosensor Selectivity

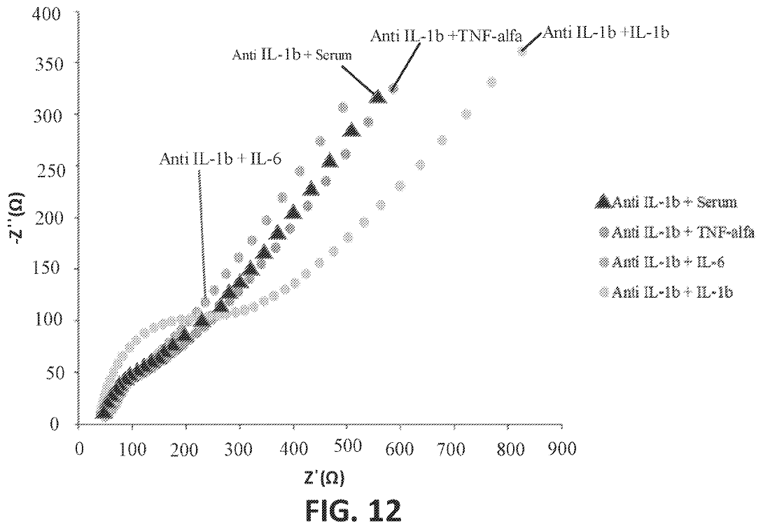

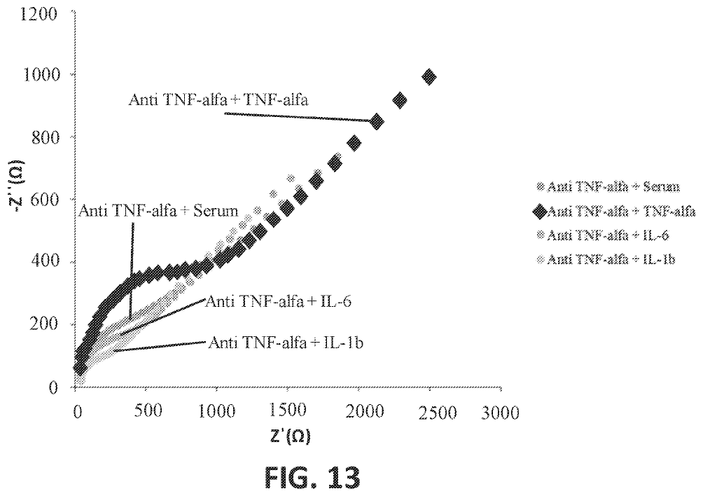

In order to determine the selectivity of each sensor, cross-reactions were tested. Nyquist impedance diagrams of carbon electrodes modified by self-assembled monolayer (SAM) with IL-1β monoclonal antibody, coupled after reaction with serum+cytokine, with a concentration of 50 pg·mL-1 for TNF-α, 10 pg·mL-1 for IL-6 and 5 pg·mL-1 for IL-1β were determined (FIG. 12). Nyquist impedance diagrams of carbon electrodes modified by SAM with monoclonal antibody anti-TNF-α, coupled after reaction with serum+cytokine, with a concentration of 50 pg·mL-1 for TNF-α, 10 pg·mL-1 for IL-6 and 5 pg·mL-1 for IL-1β were determined (FIG. 13). These results indicate that only the target cytokines responded, even when in the presence of other cytokines.

### Example 5. Detection of Sars-Cov2 S Protein

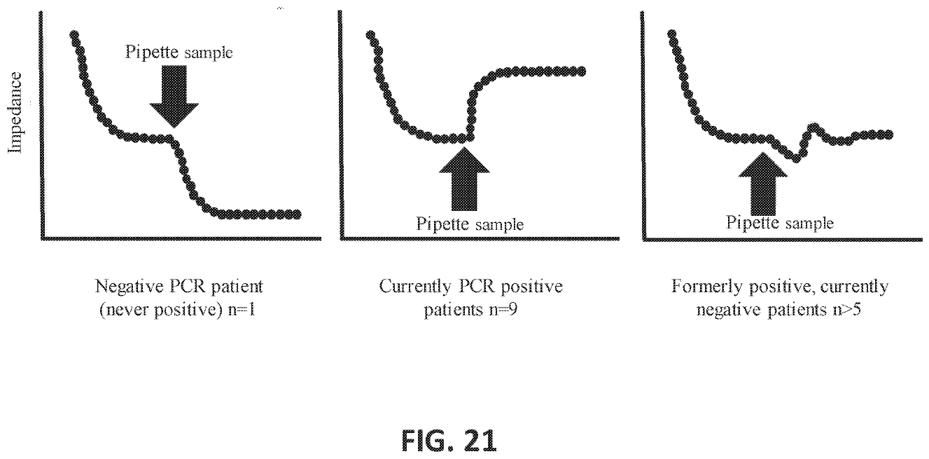

The disclosed biosensor strip was used to detect the presence of COVID-19-specific viral antigens. Forty samples from patients exposed to COVID-19 were evaluated. In the experiment shown in FIG. 21, it can be seen that the device is able to discriminate between samples which are negative for COVID-19 (verified by PCR testing) from those samples from individuals positive for COVID-19 (as verified by a PCR testing). Thus, samples from patients positive for COVID-19 displayed a distinct peak showing the presence of Sars-CoV2 Spike (S) protein. It was also found that for samples from individuals who had previously tested positive for COVID-19, but were currently negative by PCR testing and were positive by antibody testing, there was a smaller peak, likely due to a biochemical modification of the Sars-CoV2 S protein. Thus, the method provides a fingerprint by which to detect patients who are no longer actively infected, but may comprise a modified antigen (as well as anti-COVID antibody). In an embodiment, this fingerprint is diagnostic of resistance to COVID-19. This modified antigen was detected in subjects as long as several weeks to 4 months after an active infection.

### Illustrations of Suitable Methods, Devices, and Systems

Illustration A1 is a method to detect the presence of a biomarker in a subject comprising the steps of: (a) obtaining a sample from the subject; (b) applying the sample to a modified biosensor, the modified biosensor comprising: (i) a glutaraldehyde-functionalized carbon nanoparticle paste; (ii) one or more immobilized antibodies specific to the biomarker; and (iii) optionally a blocking agent; (c) applying an alternating voltage to the modified biosensor; (d) measuring an electrical impedance spectroscopy (EIS) signal to determine the presence of the biomarker.

Illustration A2 is the method of any preceding or subsequent illustration, further comprising determining a biomarker profile.

Illustration A3 is the method of any preceding or subsequent illustration, wherein the biomarker profile determines whether the subject suffers from a viral infection, bacterial infection, or inflammatory condition.

Illustration A4 is the method of any preceding or subsequent illustration, wherein the viral infection is at least one of influenza virus, herpes simplex virus (HSV), human immunodeficiency virus (HIV) type 1 (HIV-1), HIV-2 Group A, HIV-2 Group B, HIV-1 Group M, Hepatitis B, Hepatitis Delta, Ebola virus, Marburg virus, Cueva virus, West Nile Virus, Epstein-Barr Virus, Dengue Virus, adenovirus B, adenovirus C, adenovirus E, Virus, Parainfluenza Virus type 1, Parainfluenza Virus type 2, Parainfluenza Virus, Coronavirus, 229E, Coronavirus HKU1, Coronavirus OC43, Coronavirus NL63, SARS-CoV, MERS-CoV, or SARS-CoV2.

Illustration A5 is the method of any preceding or subsequent illustration, wherein the inflammatory condition is at least one of rheumatoid arthritis, multiple sclerosis, myocardial infarction, COPD, chronic nephritis, chronic hepatitis, chronic pancreatitis, Type 2 diabetes, systemic lupus erythematosus (SLE), Alzheimer's disease, Parkinson's disease (PD), or inflammatory bowel disease (IBD).

Illustration A6 is the method of any preceding or subsequent illustration, wherein the bacterial infection comprises an infection caused by at least one of Bordatella pertussis, Mycobacterium tuberculosis (MTB), Staphylococcus aureus, Methicillin-Resistant Staphylococcus aureus (MRSA), Group A Streptococcus, Group B Streptococcus, Haemophilus parainfluenzae, or Klebsiella pneumoniae.

Illustration A7 is the method of any preceding or subsequent illustration, wherein the biomarker is a viral protein or a cytokine.

Illustration A8 is the method of any preceding or subsequent illustration, wherein the viral protein is the Sars-CoV-2 S protein.

Illustration A9 is the method of any preceding or subsequent illustration, wherein the EIS signal is used to distinguish a subject with an active infection of COVID-19 from a subject in remission from a prior infection with COVID-19.

Illustration A10 is the method of any preceding or subsequent illustration, wherein the EIS signal is used to distinguish a subject with an active infection of COVID-19 from a subject who has not been exposed to COVID-19.

Illustration A11 is the method of any preceding or subsequent illustration, wherein the cytokine is a pro-inflammatory cytokine.

Illustration A12 is the method of any preceding or subsequent illustration, wherein the pro-inflammatory cytokine is at least one of tumor necrosis factor alpha (TNF-α), interleukin-1 beta (IL-1β), interleukin-6 (IL-6), interleukin-8 (IL-8), interleukin-10 (IL-10), interleukin-12 (IL-12), interferon gamma (IF-γ), CXCL10, MCP1, and MIP1α.

Illustration A13 is the method of any preceding or subsequent illustration, wherein the carbon nanoparticle paste further comprises mineral oil.

Illustration A14 is the method of any preceding or subsequent illustration, wherein the blocking agent comprises an external blocking agent such as milk or BSA.

Illustration A15 is the method of any preceding or subsequent illustration, wherein the sample is a nasal swab, blood, serum, plasma, peritoneal fluid, pleural fluid, cerebrospinal fluid, uterine fluid, saliva, or mucus.

Illustration A16 is the method of any preceding or subsequent illustration, further comprising quantifying the biomarker.

Illustration A17 is the method of any preceding or subsequent illustration, wherein no blocking agent is added, and the sample acts as a blocking agent to reduce non-specific binding.

Illustration A18 is the method of any preceding or subsequent illustration, wherein the biosensor is formulated as a strip.

Illustration B1 is a method of making a biosensor comprising: modifying a biosensor to comprise (i) a glutaraldehyde-functionalized carbon nanoparticle paste; (ii) one or more immobilized antibodies; and (iii) optionally a blocking agent.

Illustration B2 is the method of any preceding or subsequent illustration, wherein the carbon nanoparticle paste further comprises mineral oil.

Illustration B3 is the method of any preceding or subsequent illustration, wherein the blocking agent comprises an external blocking agent such as milk or BSA.

Illustration B4 is the method of any preceding or subsequent illustration, wherein the biosensor is a strip.

Illustration C1 is a biosensor device comprising: (a) a first layer comprising a carbon nanoparticle; (b) a second layer comprising glutaraldehyde; and (c) a third layer comprising one or more immobilized antibodies.

Illustration C2 is the biosensor device of any preceding or subsequent illustration, formulated as a strip

Illustration D1 is a system to detect the presence of a biomarker in an individual comprising: (a) the biosensor device of any preceding or subsequent illustration; (b) a power supply; (c) an electronic analysis device; (d) an interface; and (e) a storage device.

Illustration D2 is the system of any preceding or subsequent illustration, further comprising a computer-program product tangibly embodied in a non-transitory machine-readable storage medium, including instructions configured to run any part of the system.

Illustration E1 is a computer-program product tangibly embodied in a non-transitory machine-readable storage medium, including instructions configured to perform any of the steps of the methods of any preceding or subsequent illustration or run any part of the device of any preceding or subsequent illustration or run any part of the system of any preceding or subsequent illustration.

Illustration F1 is a method to detect antigens specific to COVID-19 in a subject comprising: (a) obtaining a sample from the subject; (b) applying the sample to a modifice biosensor, the modified biosensor comprising: (i) a glutaraldehyde-functionalized carbon nanoparticle paste; (ii) one or more immobilized antibodies specific to the biomarker; and (iii) optionally a blocking agent; (c) applying an alternating voltage to the modified biosensor; and (d) measuring an electrical impedance spectroscopy (EIS) signal to determine the presence of an antigen specific to COVID-19.

Illustration F2 is the method of any preceding or subsequent illustration, wherein the antigen is the Sars-CoV2 S protein.

Illustration F3 is the method of any preceding or subsequent illustration, further comprising detecting the presence of a modified Sars-CoV2 antigen in samples from a subject in remission for COVID-19.

Illustration F2 is the method of any preceding or subsequent illustration, wherein the subject in remission for COVID-19 is positive for a Sars-CoV2 antibody test.

Illustration F2 is the method of any preceding or subsequent illustration, wherein the sample is a nasal swab, blood, serum, plasma, peritoneal fluid, pleural fluid, cerebrospinal fluid, uterine fluid, saliva, or mucus.

Illustration F2 is the method of any preceding or subsequent illustration, wherein no blocking agent is added, and the sample acts as a blocking agent to reduce non-specific binding.

1. A method to detect the presence of a biomarker in a subject comprising the steps of:
(a) obtaining a sample from the subject;
(b) applying the sample to a biosensor, the biosensor comprising:
(i) a glutaraldehyde-functionalized carbon nanoparticle paste;
(ii) one or more immobilized antibodies specific to the biomarker; and
(iii) optionally a blocking agent;
(c) applying an alternating voltage to the biosensor;
(d) measuring an electrical impedance spectroscopy (EIS) signal to determine the presence of the biomarker.

2. The method of claim 1, further comprising determining a biomarker profile.

3. The method of claim 2, wherein the biomarker profile determines whether the subject suffers from a viral infection, bacterial infection, or inflammatory condition.

4. The method of claim 3, wherein the viral infection is at least one of influenza virus, herpes simplex virus (HSV), human immunodeficiency virus (HIV) type 1 (HIV-1), HIV-2 Group A, HIV-2 Group B, HIV-1 Group M, Hepatitis B, Hepatitis Delta, Ebola virus, Marburg virus, Cueva virus, West Nile Virus, Epstein-Barr Virus, Dengue Virus, adenovirus B, adenovirus C, adenovirus E, Virus, Parainfluenza Virus type 1, Parainfluenza Virus type 2, Parainfluenza Virus, Coronavirus, 229E, Coronavirus HKU1,
Coronavirus OC43, Coronavirus NL63, SARS-CoV, MERS-CoV, or SARS-CoV2.

5. The method of claim 3, wherein the inflammatory condition is at least one of rheumatoid arthritis, multiple sclerosis, myocardial infarction, COPD, chronic nephritis, chronic hepatitis, chronic pancreatitis, Type 2 diabetes, systemic lupus erythematosus (SLE), Alzheimer's disease, Parkinson's disease (PD), or inflammatory bowel disease (IBD).

6. The method of claim 3, wherein the bacterial infection comprises an infection caused by at least one of Bordetella pertussis, Mycobacterium tuberculosis (MTB), Staphylococcus aureus, Methicillin-Resistant Staphylococcus aureus (MRSA), Group A Streptococcus, Group B Streptococcus, Haemophilus parainfluenzae, or Klebsiella pneumoniae.

7. The method of claim 1 wherein the biomarker is a viral protein or a cytokine.

8. The method of claim 7, wherein the viral protein is the Sars-CoV-2 S protein.

9. The method of claim 8, wherein the EIS signal is used to distinguish a subject with an active infection of COVID-19 from a subject in remission from a prior infection with COVID-19.

10. The method of claim 8, wherein the EIS signal is used to distinguish a subject with an active infection of COVID-19 from a subject who has not been exposed to COVID-19.

11. The method of claim 7, wherein the cytokine is a pro-inflammatory cytokine.

12. The method of claim 8, wherein the pro-inflammatory cytokine is at least one of tumor necrosis factor alpha (TNF-α), interleukin-1 beta (IL-1β), interleukin-6 (IL-6), interleukin-8 (IL-8), interleukin-10 (IL-10), interleukin-12 (IL-12), interferon gamma (IF-γ), CXCL10, MCP1, and MIP1α.

13. The method of claim 1, wherein the carbon nanoparticle paste further comprises mineral oil.

14. (canceled)

15. (canceled)

16. The method of claim 1, further comprising quantifying the biomarker.

17. (canceled)

18. The method of claim 1, wherein the biosensor is formulated as a strip.

19. A method of making a modified biosensor comprising: modifying a biosensor to comprise: (i) a glutaraldehyde-functionalized carbon nanoparticle paste; (ii) one or more immobilized antibodies; and (iii) optionally a blocking agent.

20. The method of claim 19, wherein the carbon nanoparticle paste further comprises mineral oil.

21. (canceled)

22. The method of claim 19, wherein the biosensor is formulated as a strip.

23. A biosensor device comprising:
(a) a first layer comprising a carbon nanoparticle;
(b) a second layer comprising glutaraldehyde; and
(c) a third layer comprising one or more immobilized antibodies.

24. The biosensor of claim 23, formulated as a strip.

25. (canceled)

26. (canceled)

27. (canceled)

28. A method to detect antigens specific to COVID-19 in a subject comprising:
(a) obtaining a sample from the subject;
(b) applying the sample to a biosensor, the biosensor comprising:
(i) a glutaraldehyde-functionalized carbon nanoparticle paste;
(ii) one or more immobilized antibodies specific to the biomarker; and
(iii) optionally a blocking agent;
(c) applying an alternating voltage to the biosensor; and
(d) measuring an electrical impedance spectroscopy (EIS) signal to determine the presence of an antigen specific to COVID-19.

29. The method of claim 28, wherein the antigen is the Sars-CoV-2 S protein.

30. The method of claim 28, further comprising detecting the presence of a modified Sars-CoV2 antigen in samples from a subject in remission for COVID-19.

31. The method of claim 30, wherein the subject in remission for COVID-19 is positive for a Sars-CoV2 antibody test.

32. (canceled)

33. (canceled)
